(19) United States
(12) Patent Application Publication
(10) Pub. No.: US 2026/0162828 A1
(43) Pub. Date: Jun. 11, 2026

(54) METHODS AND TOOLS FOR ASSESSING CARDIOMETABOLIC HEALTH AND HIDDEN DISEASE RISK AMONG APPARENTLY HEALTHY INDIVIDUALS

(71) Applicant: Texas Tech University System, Lubbock, TX (US)

(72) Inventors: David P. Cistola, El Paso, TX (US); Alok K. Dwivedi, El Paso, TX (US)

(73) Assignee: Texas Tech University System, Lubbock, TX (US)

(21) Appl. No.: 19/126,991

(22) PCT Filed: Nov. 6, 2023
(86) PCT No.: PCT/US2023/036846

§ 371 Date: May 2, 2025

Related U.S. Application Data
(60) Provisional application No. 63/422,948, filed on Nov. 5, 2022.

(51) Int. Cl. G16H 50/30

(52) U.S. Cl. CPC G16H 50/30

(57) ABSTRACT

Embodiments of the present disclosure pertain to methods and systems for assessing a subject's vulnerability to developing at least one cardiometabolic-related condition. Such methods and systems generally include the following steps or instructions: (1) receiving a plurality of health-related data of the subject; (2) calculating a risk score from the plurality of health-related data; (3) correlating the risk score to the subject's vulnerability to the cardiometabolic-related condition; and (4) making a treatment decision based on the subject's vulnerability to the cardiometabolicrelated condition.

### CROSS-REFERENCE TO RELATED APPLICATIONS

This application claims priority to U.S. Provisional Patent Application No. 63/422,948, filed on Nov. 5, 2022. The entirety of the aforementioned application is incorporated herein by reference.

### STATEMENT REGARDING FEDERALLY SPONSORED RESEARCH

This invention was made with government support under R21 HL143030 awarded by the National Institutes of Health. The government has certain rights in the invention.

### BACKGROUND

A need exists for the early detection of abnormal or unbalanced metabolism. Numerous embodiments of the present disclosure aim to address the aforementioned need.

### SUMMARY

In some embodiments, the present disclosure pertains to methods of assessing a subject's vulnerability to developing at least one cardiometabolic-related condition. In some embodiments, such methods include: (1) receiving a plurality of health-related data of the subject; (2) calculating a risk score from the plurality of health-related data; and (3) correlating the risk score to the subject's vulnerability to the cardiometabolic-related condition. In some embodiments, the methods of the present disclosure also include a step of (4) making a treatment decision based on the subject's vulnerability to the cardiometabolic-related condition. In some embodiments, the method is repeated after implementing the treatment decision.

Additional embodiments of the present disclosure pertain to systems for assessing a subject's vulnerability to developing at least one cardiometabolic-related condition. In some embodiments, such systems include: (1) instructions for receiving a plurality of health-related data of the subject; (2) instructions for calculating a risk score from the plurality of health-related data; and (3) instructions for correlating the risk score to the subject's vulnerability to the cardiometabolic-related condition. In some embodiments, the systems of the present disclosure also include (4) instructions for making a treatment decision based on the subject's vulnerability to the cardiometabolic-related condition. In some embodiments, the systems of the present disclosure also include (5) instructions for repeating the aforementioned instructions after implementing the treatment decision.

### DESCRIPTION OF THE DRAWINGS

FIG. 1A provides an illustration of a method of assessing a subject's vulnerability to developing at least one cardiometabolic-related condition.

FIG. 1B provides an illustration of a computing device for assessing a subject's vulnerability to developing at least one cardiometabolic-related condition.

FIG. 2A provides an illustration of early metabolic imbalance (EMI), a hidden state of compensated insulin resistance (IR).

FIG. 2B provides an illustration of the progression of EMI to type 2 diabetes.

FIG. 3 shows Kaplan-Meier survival curves for incident diabetes, by insulin-BMI category, unadjusted, with 30-year follow up. This was a retrospective cohort analysis of the Coronary Artery Risk Development in Young Adults (CARDIA) study, where the study population at baseline (n=3,292) included 18-30 year olds with fasting glucose, fasting triglycerides and high-density lipoprotein cholesterol (HDL-C) all within normal limits (no prediabetes, metabolic syndrome, diabetes or CVD at baseline). High insulin was defined as 7.5 μIU/mL for individuals with BMI <25 and 11.8 μIU/mL for individuals with BMI ≥25. Insulin cut points were calibrated using time-dependent receiver operator characteristic curve (time ROC) analyses.

FIG. 4 shows a forest plot with hazard ratios, 95% confidence intervals (CI) and p-values for 8 combinations of the 3-way interaction between fasting insulin, waist circumference (WC) and fasting glucose. Values were derived from a Cox proportional hazards regression model for incident cardiovascular disease (CVD) using the CARDIA n=3,292 cohort mentioned in FIG. 3; covariates: 2019 ACC/AHA risk factors for CVD. The Harrell's c-statistic for this model was 0.728; as a general rule, models with c≥0.700 are considered to have acceptable prognostic capability.

FIG. 5 shows a forest plot displaying hazard ratios for a Cox proportional hazard regression model essentially identical to that shown in FIG. 4, except that it also contained high GGT and high platelet count (marker of inflammation) as additional covariates. GGT, gamma glutamyl transferase. The addition of these two predictors increased the Harrell's c-statistic to 0.740.

FIG. 6 shows a classification tree for CVD risk assessment in apparently healthy young adults from CARDIA (i.e., those without metabolic syndrome or prediabetes at baseline). The relative risk values (hazard ratios) shown at the bottom were derived from the Cox Model shown in FIG. 5 for the CARDIA subpopulation where n=3,292, as defined in FIG. 3. Prognostic cut points were calibrated using time-ROC analysis: high-normal glucose=82-99 mg/dL, low-normal <82 mg/dL; high WC=80.75 cm (male) or 72.25 (female); high insulin=12.1 μIU/mL for the high-normal glucose/high WC stratum; 9.6 μIU/mL for low-normal glucose/high WC stratum; 9.1 μIU/mL for high-normal glucose/low WC stratum; 6.3 μIU/mL for low-normal glucose/low WC stratum, where insulin ≥6.3 μIU/mL in that stratum is protective against CVD. Relative risk values highlighted in red are statistically higher than the reference group (1.0).

FIG. 7 shows unadjusted Kaplan-Meier curves, with 95% confidence interval bands, for incident CVD, by fasting insulin tertiles, from a retrospective cohort analysis of the CARDIA subpopulation defined in FIG. 3, where n=3,292.

FIG. 8 shows the overall time-ROC curve for fasting insulin as a prognostic indicator of incident CVD, without stratification by BMI or glucose. The covariate-adjusted time ROC curve (not shown) yielded a similar cut point: 9.1 μIU/mL. The cumulative incidence of CVD during the 34-year follow up period was 5.29% (174 cases).

FIGS. 9A-9D show time-ROC curves for fasting insulin as a prognostic indicator of incident CVD, stratified by BMI & glucose categories.

FIG. 10 shows a forest plot displaying Cox hazard ratios, 95% confidence intervals and p-values for 8 combinations of the 3-way interaction between fasting insulin, BMI and fasting glucose. This Cox model for incident CVD also includes the 2019 canonical ACC/AHA risk factors (not shown). The prognostic power, as measured by the Harrell's c-statistic, is 0.721. As a general rule, models with a c-statistic >0.700 are considered to have acceptable prognostic power.

FIG. 11 shows Kaplan-Meier curves, with 95% confidence interval bands, for incident diabetes with 30-year follow up. This retrospective cohort analysis was performed with the CARDIA subpopulation defined in FIG. 3.

FIG. 12 shows an unadjusted time-ROC curve for the original 1985 fasting insulin assay (where the antibody cross-reacted with proinsulin) as a prognostic indicator of incident diabetes. There was no adjustment for covariates. The cumulative incidence of diabetes at 360 months was 10.9% (359 cases). This analysis was performed with the CARDIA subpopulation defined in FIG. 3.

FIG. 13 shows a covariate-adjusted time-ROC curve for the original 1985 fasting insulin assay. This analysis was adjusted for family history of diabetes, race, sex, age, fasting glucose, physical fitness, hypertension, serum cotinine, uric acid, white blood cells (WBC), and triglyceride (TG)/HDL-cholesterol ratios. However, the results were essentially identical to the unadjusted analysis shown in FIG. 12. This analysis was performed with the CARDIA subpopulation defined in FIG. 3.

FIGS. 14A-14B show time-ROC curves for the original 1985 fasting insulin assay as a prognostic indicator for incident diabetes, stratified by BMI (<25, FIG. 14A, vs ≥25, FIG. 14A), and adjusted for covariates as listed in FIG. 13.

FIG. 15 shows an overall time-ROC curve for the newer, more specific 1992 fasting insulin assay (where the antibody did not cross react with pro-insulin). This analysis was adjusted for covariates as listed in FIG. 13.

FIGS. 16A-16B show time-ROC curves for the newer 1992 fasting insulin assay, as stratified by BMI (<25, FIG. 16A vs ≥25, FIG. 16B) and adjusted for the covariates listed in FIG. 13.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

The early detection of abnormal or unbalanced metabolism is essential for identifying and estimating an individual's risk for future cardiometabolic diseases and for preventing those diseases. For instance, early metabolic imbalance (EMI) is an overlooked condition that puts apparently healthy people at increased risk for future type 2 diabetes and cardiovascular disease (CVD). Thus, EMI may be a hidden early stage in the development of cardiometabolic diseases and their preconditions, such as prediabetes and metabolic syndrome.

EMI includes an interplay between early compensated insulin resistance, hyperinsulinemia, subclinical inflammation, hypoxia, oxidative stress and pro-coagulation. This hidden condition is prevalent, affecting approximately 9.4% of the U.S. population ages 12 and up, or 26 million people.

EMI is especially prevalent in young people, including approximately 20% of teenagers and 15% of young adults ages 20-29. Most healthcare providers are unaware of EMI. EMI evades conventional screening performed by medical offices, since blood glucose and lipids are within normal limits. Thus, individuals with EMI do not meet the criteria for prediabetes or metabolic syndrome and are incorrectly viewed by providers as “low risk.”

In fact, most physicians and health care providers are unaware of EMI and its impact on future disease risk. Current medical practice standards do not mention or address this condition. Thus, few physicians screen for EMI.

Measurements for detecting EMI, when properly combined and interpreted, are currently available. However, there are no easy-to-use screening tools for clinical providers or the general public that combine and interpret these measurements in the proper way to assess cardiometabolic health and disease risk.

Additionally, most providers view insulin resistance, subclinical inflammation, subclinical tissue hypoxia and oxidative stress as underlying components of prediabetes and metabolic syndrome, rather than a condition that can occur earlier. For those rare providers who screen for early insulin resistance, they may use a fasting insulin or fasting c-peptide blood test, either alone or as input into the Homeostatic Model Assessment of Insulin Resistance (HOMA2-IR) calculator. Alternatively, they may use an expanded oral glucose tolerance test with measurements of both blood insulin and glucose before and after the ingestion of a test drink containing a fixed amount of sugar. Using these measures, insulin resistance can be estimated using the Matsuda Index. These assessments are rarely used in the clinic setting, especially in individuals who have normal fasting glucose or hemoglobin A1c.

The normal reference ranges for fasting insulin and c-peptide do not take EMI into consideration. As such, individuals with the condition are often missed. Even if the clinician uses properly calibrated cut points, the sensitivity of insulin and HOMA2-IR for detecting early diabetes and CVD risk is only ˜60%, leaving a 40% false negative rate. Other available alternatives are the American Diabetes Association risk test for type 2 diabetes. However, this calculator is not calibrated for EMI and does not detect individuals with EMI.

Similar risk calculators are available for CVD. However, such calculators are not calibrated for EMI.

The aforementioned limits for the early detection of abnormal or unbalanced metabolism represent an unmet medical need. For instance, the key to the prevention of type 2 diabetes is to preserve the pancreatic beta cells that secrete insulin into the bloodstream. By the time individuals develop prediabetes (i.e., impaired glucose tolerance), a 50-70% decline in beta cell function has already occurred, without symptoms.

Similarly, the key to preventing atherosclerotic cardiovascular disease (ASCVD) is to preserve the integrity of the arterial wall, the lining of vessels that supply blood to vital organs like the heart and brain. By the time individuals develop metabolic syndrome, insidious arterial plaque development and damage has already occurred.

As such, a need exists for better methods and tools to screen for early cardiometabolic imbalance and estimate risk for future disease. In particular, a need exists for healthcare providers and the general public to screen for cardiometabolic health and estimate future disease risk in apparently healthy individuals. Numerous embodiments of the present disclosure address the aforementioned needs.

### Methods and Systems for Assessing Vulnerability to Cardiometabolic-Related Conditions

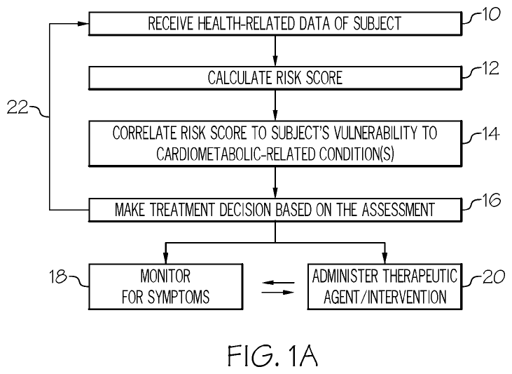

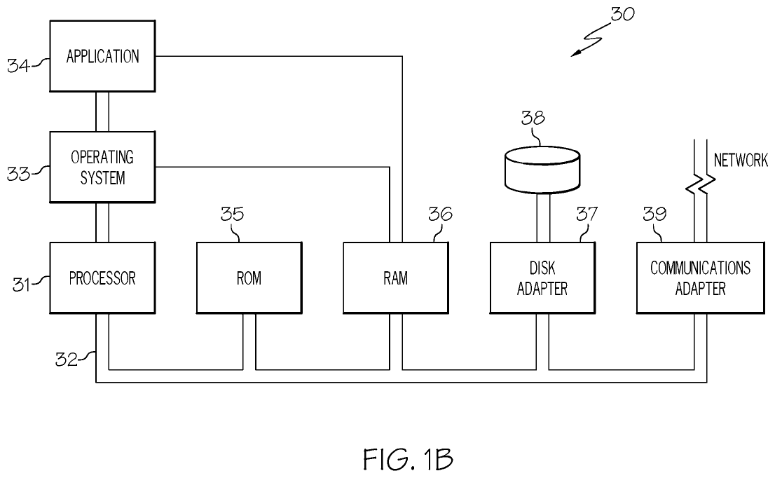

In some embodiments, the present disclosure pertains to methods of assessing a subject's vulnerability to developing at least one cardiometabolic-related condition. In some embodiments illustrated in FIG. 1A, such methods include: receiving a plurality of health-related data of the subject (step 10); calculating a risk score from the plurality of health-related data (step 12); and correlating the risk score to the subject's vulnerability to the cardiometabolic-related condition (step 14). In some embodiments, the methods of the present disclosure also include a step of making a treatment decision based on the subject's vulnerability to the cardiometabolic-related condition (step 16). In some embodiments, the treatment decision includes monitoring the subject for signs or symptoms of the cardiometabolic-related condition or its pre-conditions (step 18), and/or administering a therapeutic agent and/or therapeutic intervention to the subject (step 20). In some embodiments, the method is repeated after implementing the treatment decision (step 22).

Additional embodiments of the present disclosure pertain to systems for assessing a subject's vulnerability to developing at least one cardiometabolic-related condition. In some embodiments, such systems include: (1) instructions for receiving a plurality of health-related data of the subject; (2) instructions for calculating a risk score from the plurality of health-related data; and (3) instructions for correlating the risk score to the subject's vulnerability to the cardiometabolic-related condition. In some embodiments, the systems of the present disclosure also include (4) instructions for making a treatment decision based on the subject's vulnerability to the cardiometabolic-related condition. In some embodiments, the systems of the present disclosure also include (5) instructions for repeating the aforementioned instructions after implementing the treatment decision. As set forth in more detail herein, the methods and systems of the present disclosure can have numerous embodiments.

### Health-Related Data

The methods and systems of the present disclosure may utilize various types of health-related data. For instance, in some embodiments, the health-related data include, without limitation, demographic data, age, gender assigned at birth, race, socio-economic history, medical history, family history of diabetes, family history of cardiovascular disease, clinical data, vital signs, laboratory test results, blood test results, imaging test results, non-invasive health measurements, body-mass index (BMI), waist circumference, waist circumference-to-height ratio, fitness level, Fitbit activity, electrocardiogram (EKG) profile, pulse oximetry data, blood gas parameters (e.g., % oxy-hemoglobin, % deoxy-hemoglobin, % met-hemoglobin and/or % oxidized hemoglobin), blood pressure, hypertension, body composition measurements, inflammatory marker levels, C-reactive protein levels, platelet levels, white blood cell (WBC) levels, oxidative stress marker levels, tissue hypoxia marker levels, gamma glutamyl transferase (GGT) levels, aspartate aminotransferase (AST) levels, alanine aminotransferase (ALT) levels, bilirubin levels, lactic acid levels, lactate levels, uric acid levels, urate levels, uric acid/creatinine ratio, cotinine levels, insulin resistance measures, beta cell insulin secretion levels, insulin levels, fasting insulin levels, non-fasting insulin levels, fasting c-peptide levels, non-fasting c-peptide levels, glucose levels, fasting glucose levels, non-fasting glucose levels, hemoglobin A1c levels, triglyceride (TG) levels, fasting TG levels, non-fasting TG levels, high-density lipoprotein cholesterol (HDL-C) levels, TG/HDL ratio, low-density lipoprotein cholesterol (LDL-C) levels, oxidized LDL-C levels, non-HDL-C levels (i.e., HDL-C levels subtracted from total cholesterol levels), apolipoprotein B levels, apolipoprotein C-III levels, physical fitness measures, and combinations thereof.

In some embodiments, the plurality of health-related data include, without limitation, fasting insulin levels, non-fasting insulin levels, fasting c-peptide levels, non-fasting c-peptide levels, fasting glucose levels, non-fasting glucose levels, hemoglobin A1c levels, fasting triglyceride levels, non-fasting triglyceride levels, high-density lipoprotein (HDL) levels, and combinations thereof. In some embodiments, the plurality of health-related data include fasting or non-fasting insulin levels, c-peptide levels, and glucose levels.

In some embodiments, the plurality of health-related data include, without limitation, age, gender assigned at birth, race, fasting insulin levels, fasting c-peptide levels, insulin resistance measures, fasting glucose levels, beta cell insulin secretion levels, fasting triglyceride (TG) levels, high-density lipoprotein cholesterol (HDL-C) levels, TG/HDL ratio, low-density lipoprotein cholesterol (LDL-C) levels, uric acid levels, platelet levels, white blood cell (WBC) levels, cotinine levels, gamma glutamyl transferase (GGT) levels, body-mass index (BMI), fitness level, hypertension, family history of diabetes, family history of cardiovascular disease, waist circumference, waist circumference-to-height ratio, and combinations thereof. In some embodiments, the plurality of health-related data include fasting insulin or fasting c-peptide levels, fasting glucose or hemoglobin A1c levels, cotinine levels, body-mass index (BMI), fitness level, hypertension, and family history of diabetes.

In some embodiments, the plurality of health-related data include fasting insulin or fasting c-peptide levels, fasting glucose or hemoglobin A1c levels, and waist circumference or body-mass index (BMI). In some embodiments, the plurality of health-related data include fasting insulin or fasting c-peptide levels, fasting glucose or hemoglobin A1c levels, waist circumference or body-mass index (BMI) or waist circumference-to-height ratio, platelet levels, and gamma glutamyl transferase (GGT) levels.

In some embodiments, the plurality of health-related data include fasting insulin or fasting c-peptide levels, fasting glucose or hemoglobin A1c levels, and body-mass index (BMI) or wait circumference or waist circumference-to-height ratio. In some embodiments, the plurality of health-related data include family history of diabetes, race, gender assigned at birth, age, fasting glucose or hemoglobin A1c levels, fasting insulin or fasting c-peptide levels, physical fitness measures, hypertension, cotinine levels, uric acid levels, TG/HDL ratio, and white blood cell (WBC) levels.

### Measuring or Obtaining Health-Related Data

In some embodiments, the methods and systems of the present disclosure also include a step of, or instructions for, measuring or obtaining a plurality of health-related data. In some embodiments, the health-related data may be measured or obtained from a tissue sample, a body fluid, a blood sample, or a non-invasive recording of a subject. In some embodiments, the methods and systems of the present disclosure also include a step of, or instructions for, obtaining a tissue sample, body fluid, blood sample, or a non-invasive recording from a subject and measuring a plurality of health-related data from the tissue sample, body fluid, blood sample or non-invasive recording.

### Risk Score Calculation and Correlation

The methods and systems of the present disclosure may be utilized to calculate risk scores from health-related data in various manners. The methods and systems of the present disclosure may also be utilized to correlate calculated risk scores to a subject's vulnerability to a cardiometabolic-related condition in various manners. For instance, in some embodiments, a risk score may be calculated based on a subject's fasting insulin or fasting c-peptide level and fasting glucose or hemoglobin A1c levels. In some embodiments, a risk score may represent calculated hazard ratios, which include a measure of future disease risk. In some embodiments, a risk score may represent a cardiometabolic health and risk score.

The following methods (i.e., steps 1-8 presented herein) provide examples on how to develop and estimate the state of cardiometabolic health and a subject's vulnerability to cardiometabolic-related conditions from a target population. The risk score calculations can be performed using novel equations built into simple-to-use phone and computer apps, web-based calculators, clinical lab reports, electronic medical records and nomograms.

The steps 1-8 presented herein describe how risk score equations are developed, calibrated, validated and implemented. The steps 1-8 presented herein may be followed for each cardiometabolic-related condition for which risk is to be assessed (e.g., prediabetes, type 2 diabetes, metabolic syndrome, and/or atherosclerotic cardiovascular disease (ASCVD)).
Step 1: Select each target population for risk assessment. Using data from a suitable longitudinal cohort study with appropriate start date and duration, select the target population. For illustration, the Coronary Artery Risk Development in Young Adults (CARDIA) cohort study began in 1985 with 5,114 young adults ages 18-30 years at baseline. The participants completed comprehensive health questionnaires and medical examinations, including blood tests, both at baseline and during subsequent follow up over the past 35 years. After excluding participants who had prediabetes, diabetes, metabolic syndrome or ASCVD at baseline, the target population of subjects ages 18-30 is established. By shifting the “baseline” to 1992, a new target population of healthy subjects ages 25-37 can be used. Other cohort studies can be used as well to capture different target populations of apparently healthy subjects.
Step 2: Calibrate fasting insulin cut points for each target population. Using conventional and time-dependent receiver operator characteristic curve (logROC and timeROC) analyses, calibrate the prognostic cut point optimal for fasting insulin for incident disease (e.g., diabetes or ASCVD) or precondition (e.g., prediabetes or metabolic syndrome). Because of the interaction between insulin and measures of obesity, the cut point analyses are stratified by BMI or waist circumference. For example, stratification by BMI would yield different insulin cut points for subjects with BMI <25 (normal weight) vs. >=25 (overweight or obese).
Step 3: Identify hidden risk markers in the target population. Using data from the National Health and Nutrition Examination Survey (NHANES), which represents the U.S. population, divide the participants with/without prediabetes, metabolic syndrome, diabetes or ASCVD into separate groups. Then further divide the “without” group into those with high and low fasting insulin using the cut points calibrated in Step 2. Using multivariable logistic regression analysis, identify covariates (variables, factors or combinations) that have statistically significant association with high fasting insulin in otherwise healthy subjects. Possible examples may include markers of subclinical inflammation, oxidative stress, cell & tissue damage, pro-coagulation, hypoxemia and/or hypoxia.
Step 4: Determine whether the insulin covariates from Step 3 are risk factors by assessing their prognostic value. Using suitable cohort data and Cox proportional hazards regression, generate a series of Cox statistical models. This process starts with a base model consisting of previously established “canonical” risk factors only. The second model replaces BMI or waist circumference (WC) category with a combined variable containing both insulin and BMI/WC category. Each subsequent model adds a new candidate risk factor from Step 3. For each model, the Cox hazard ratio and Harrell's c-statistic are estimated to assess association and discriminatory abilities for incident disease. This process is iterated until a set of risk factors that provides the maximum prognostic value is identified.
Step 5: Define and calibrate the risk equations. There are several general strategies for risk score development, which have varying degrees of performance. The tradeoff is between simplicity and accessibility as opposed to prediction accuracy. The best performance accuracy can be achieved using equations derived from properly executed Cox proportional hazards regression. Such analyses incorporate the optimal combination of risk factors in their correct form and account for the proportional hazards and linearity assumptions of Cox regression. Using the input data and the regression equations/coefficients, a quantitative estimate of health status and disease risk can be obtained.

Additionally, simpler approaches with less prediction accuracy can be developed without the need for equations. These strategies are based on simple approximations of the risk terms, yielding crude, less accurate risk estimates. Other, more complex approaches use machine learning to systematically search for optimal combinations of risk terms and regression equations. In some embodiments, Applicant's methods test all of these approaches to assess and compare their prediction accuracy for each disease and target population of interest.
Step 6: Validate the risk equations. In this step, the equations are validated internally or externally. International validation uses the bootstrap or leave one out method to see if the model consistently produces a similar Harrell's c-statistic. External validation assesses whether the method can be used in different populations without losing its performance.

The external validation may be performed in two ways. In the first approach, the cohort population is randomly divided into a 60:40 ratio. A regression model is then developed in the training set (60%) and validated using the test set (other 40%). This process is repeated 5-10 times, and the c-statistics are compared. The second approach to external validation uses completely different cohort studies for model development and validation. In preferred embodiments, Applicant's strategy utilizes internal validation, followed by external validation.
Step 7: Naming the tools for clinical and non-clinical use and understanding: In some embodiments, in order to promote ease of use and avoid confusion, it is preferable to properly name and trademark the tools to promote their proper use and understanding in real-world settings.
Step 8: Incorporating the risk equations into cell phone and computer apps, web-based calculators, clinical laboratory test reports, medical records and nomograms for dissemination and public use. In some embodiments, it is likely that optimized computer algorithms (as opposed to crude manual estimates) may be needed to achieve optimal prediction accuracy. Given the wide availability of cell phones and computers, this is not a major obstacle. In some embodiments, an end user may enter the input data into a cell phone or computer app and quickly obtain a report with health assessment and risk estimates. In another embodiment, a web-based calculator may be used, without a need for installing a phone or computer app. In another embodiment, the equations could be built into clinical laboratory test reports, so that providers and subjects (e.g., patients) would receive the results automatically without having to input the data. In another embodiments, the equations could be incorporated into electronic medical records, likewise providing an automated report. In another embodiment, a nomogram can be supplemented alongside of risk equations so that one can use the graphic approach to compute their own risk for disease.

### Cardiometabolic-Related Conditions

Risk scores may be correlated to a subject's vulnerability to various types of cardiometabolic-related conditions. For instance, in some embodiments, the cardiometabolic-related condition includes, without limitation, early metabolic imbalance (EMI), prediabetes, hyperinsulinemia, compensatory hyperinsulinemia, metabolic syndrome, insulin resistance syndrome, early metabolic dysregulation, diabetes, type 2 diabetes, gestational diabetes, latent autoimmune diabetes of adults, monogenic forms of diabetes, type 1 diabetes, cardiovascular disease (CVD), atherosclerotic cardiovascular disease (ASCVD), heart attack, stroke, peripheral vascular disease, insulin resistance, subclinical inflammation, oxidative stress, hypoxemia, subclinical hypoxemia, hypoxia, subclinical hypoxia, pre-conditions thereof, and combinations thereof.

In some embodiments, the cardiometabolic-related condition represents a pre-condition of the cardiometabolic-related condition. In some embodiments, the cardiometabolic-related condition includes early metabolic imbalance (EMI). In some embodiments, the cardiometabolic-related condition includes cardiovascular disease (CVD). In some embodiments, the cardiometabolic-related condition includes atherosclerotic cardiovascular disease (ASCVD). In some embodiments, the cardiometabolic-related condition includes forms of diabetes, particularly type 2 diabetes, gestational diabetes, latent autoimmune diabetes of adults, monogenic forms of diabetes and type 1 diabetes

### Treatment Decisions

In some embodiments, the methods of the present disclosure also include a step of making a treatment decision based on a subject's vulnerability to a cardiometabolic-related condition. Similarly, in some embodiments, the systems of the present disclosure include instructions for making a treatment decision based on a subject's vulnerability to a cardiometabolic-related condition.

In some embodiments, the treatment decision includes monitoring the subject for signs or symptoms of a cardiometabolic-related condition, monitoring the subject for pre-conditions or risk factors of a cardiometabolic-related condition, administering a therapeutic agent to the subject, and combinations thereof. In some embodiments, the treatment decision includes monitoring the subject for signs or symptoms of a cardiometabolic-related condition. In some embodiments, the treatment decision includes monitoring the subject for pre-conditions or risk factors of a cardiometabolic-related condition.

In some embodiments, the treatment decision includes administering a therapeutic agent or intervention to the subject. In some embodiments, the therapeutic intervention includes, without limitation, a nutritional program, a physical activity program, a weight-loss program, a non-pharmaceutical intervention, administration of one or more pharmaceutical agents (e.g., anti-obesity medications), administration of one or more nutritional supplements, and combinations thereof.

In some embodiments, the methods of the present disclosure may be repeated after implementing a treatment decision. Similarly, in some embodiments, the systems of the present disclosure may include instructions for repeating prior instructions after implementing the treatment decision.

### Subjects

The methods and systems of the present disclosure may be utilized to assess the vulnerability of various subjects to cardiometabolic-related conditions. For instance, in some embodiments, the subject is a human being.

In some embodiments, the subject is a healthy subject. In some embodiments, the subject is not suffering from, or diagnosed with, a cardiometabolic-related condition to be assessed. In some embodiments, the subject is not suffering from or diagnosed with prediabetes, diabetes, metabolic syndrome, cardiovascular disease, hyperglycemia, hypertriglyceridemia, or low HDL cholesterol. In some embodiments, the subject has normal levels of fasting glucose, hemoglobin A1c, fasting triglycerides, and HDL cholesterol.

The methods and systems of the present disclosure may be applied to subjects of various age groups. For instance, in some embodiments, the subject is less than 50 years of age. In some embodiments, the subject is less than 40 years of age. In some embodiments, the subject is less than 30 years of age. In some embodiments, the subject is less than 20 years of age. In some embodiments, the subject is less than 18 years of age.

### Modes of Operation

The methods and systems of the present disclosure may be operated in various manners. For instance, in some embodiments, the methods and systems of the present disclosure operate manually. In some embodiments, the methods and systems of the present disclosure do not involve the use of a computing device.

For instance, in some embodiments, the methods of the present disclosure occur through utilization of a manual health and risk score calculator. Similarly, in some embodiments, instructions associated with the systems of the present disclosure include a manual health and risk score calculator. In some embodiments, the manual health and risk score calculator is in the form of a fillable questionnaire. In some embodiments, the manual health and risk score calculator is developed through the utilization of a simple risk and health score questionnaire from statistical analysis of human population data.

In some embodiments, the methods and systems of the present disclosure operate automatically. For instance, in some embodiments, the methods and systems of the present disclosure involve the utilization of a computing device.

In some embodiments, the systems of the present disclosure include a computer-implemented system where the system's instructions include programming instructions of a computing device. In particular embodiments, the systems of the present are in the form of a computer-implemented system that include: programming instructions for receiving a plurality of health-related data of a subject; programming instructions for calculating a risk score from the plurality of health-related data; and programming instructions for correlating the risk score to the subject's vulnerability to a cardiometabolic-related condition.

In some embodiments, the computer-implemented systems of the present disclosure also include programming instructions for making a treatment decision based on a subject's vulnerability to a cardiometabolic-related condition. In some embodiments, the computer-implemented systems of the present disclosure also include programming instructions for repeating the aforementioned instructions after implementing the treatment decision.

In some embodiments, the methods of the present disclosure occur through the utilization of a computing device. In some embodiments, the step of receiving a plurality of health-related data includes entering the health-related data to the computing device. In some embodiments, the step of calculating a risk score occurs by the computing device. In some embodiments, the step of correlating the risk score to the subject's vulnerability to a cardiometabolic-related condition includes generating an output from the computing device. In some embodiments, a step of making a treatment decision based on a subject's vulnerability to a cardiometabolic-related condition also includes generating an output from the computing device.

The methods and systems of the present disclosure may utilize various types of computing devices. For instance, in some embodiments, the computing device includes a web-based program, an application-based program, and combinations thereof.

In some embodiments, the computing device includes an artificial intelligence algorithm trained on a plurality of health-related data. In some embodiments, the computing device includes a machine-learning algorithm trained on a plurality of health-related data. In some embodiments, the step of, or instructions for, receiving a plurality of health-related data include feeding the health-related data to the machine-learning algorithm. In some embodiments, the step of, or instructions for calculating a risk score occur by the machine-learning algorithm. In some embodiments, the step of, or instructions for, correlating the risk score to the subject's vulnerability to a cardiometabolic-related condition includes generating an output from the machine-learning algorithm.

The methods and systems of the present disclosure may utilize various types of machine learning algorithms. For instance, in some embodiments, the machine-learning algorithm is an L₁-regularized logistic regression algorithm. In some embodiments, the machine-learning algorithm is a machine learning algorithm trained on a plurality of health-related data. In some embodiments, the machine learning algorithm includes supervised learning algorithms. In some embodiments, the supervised learning algorithms include nearest neighbor algorithms, naïve-Bayes algorithms, decision tree algorithms, linear regression algorithms, support vector machines, neural networks, convolutional neural networks, ensembles (e.g., random forests and gradient-boosted decision trees), and combinations thereof.

The machine learning algorithms of the present disclosure may be trained in various manners. For instance, in some embodiments, the training includes (1) feeding a plurality of health-related data into a machine learning algorithm, where the health-related data are from one or more subjects that have or have not developed one or more cardiometabolic-related conditions; (2) feeding another set of health-related data into the machine learning algorithm, where the health-related data are from one or more subjects that have or have not developed one or more cardiometabolic-related conditions; and (3) training the machine learning algorithm to assess a subject's vulnerability to one or more cardiometabolic-related conditions by comparing the aforementioned categories of health-related data. In some embodiments, training a machine learning algorithm includes adjusting weights or parameters within the machine learning algorithm to differentiate between the aforementioned categories of health-related data. In some embodiments, training a machine learning algorithm includes providing health-related data relevance values to differentiate between the aforementioned categories.

### Computing Device Architectures

Computing devices used in the present disclosure can have numerous variations and architectures. For instance, the computing devices of the present disclosure can include various types of computer-readable storage mediums. In some embodiments, the computer-readable storage mediums can be a tangible device that can retain and store instructions for use by an instruction execution device. In some embodiments, the computer-readable storage medium may include, without limitation, an electronic storage device, a magnetic storage device, an optical storage device, an electromagnetic storage device, a semiconductor storage device, and combinations thereof. A non-exhaustive list of more specific examples of suitable computer-readable storage medium includes, without limitation, a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device, and combinations thereof.

A computer-readable storage medium, as used herein, is not to be construed as being transitory signals per se. Such transitory signals may be represented by radio waves or other freely propagating electromagnetic waves, electromagnetic waves propagating through a waveguide or other transmission media (e.g., light pulses passing through a fiber-optic cable), or electrical signals transmitted through a wire.

In some embodiments, computer-readable program instructions described herein can be downloaded to respective computing/processing devices from a computer-readable storage medium or to an external computer or external storage device via a network, such as the Internet, a local area network, a wide area network and/or a wireless network. In some embodiments, the network may include copper transmission cables, optical transmission fibers, wireless transmission, routers, firewalls, switches, gateway computers and/or edge servers. In some embodiments, a network adapter card or network interface in each computing/processing device receives computer-readable program instructions from the network and forwards the computer-readable program instructions for storage in a computer-readable storage medium within the respective computing/processing device.

In some embodiments, computer-readable program instructions for carrying out operations of the present disclosure may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object-oriented programming language such as Smalltalk, C++, or the like, and procedural programming languages, such as the “C” programming language or similar programming languages.

In some embodiments, the computer-readable program instructions may execute entirely on the user's computer as a stand-alone software package, partly on the user's computer and partly on a remote computer or entirely on the remote computer or server. In the latter scenario, the remote computer may be connected in some embodiments to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer-readable program instructions by utilizing state information of the computer-readable program instructions to personalize the electronic circuitry in order to perform aspects of the present disclosure.

Embodiments of the present disclosure as discussed herein may be implemented using a computing device illustrated in FIG. 1B. Referring now to FIG. 1B, FIG. 1B illustrates an embodiment of the present disclosure of the hardware configuration of a computing device 30 represents a hardware environment for practicing various embodiments of the present disclosure.

Computing device 30 has a processor 31 connected to various other components by system bus 32. An operating system 33 runs on processor 31 and provides control and coordinates the functions of the various components of FIG. 1B. An application 34 in accordance with the principles of the present disclosure runs in conjunction with operating system 33 and provides calls to operating system 33, where the calls implement the various functions or services to be performed by application 34. Application 34 may include, for example, a program for assessing a subject's vulnerability to developing at least one cardiometabolic-related condition, such as in connection with FIGS. 1A, 4-6, 8, 9A-9D, 10, 12-13, 14A-14B, 15, and 16A-16B.

Referring again to FIG. 1B, read-only memory (“ROM”) 35 is connected to system bus 32 and includes a basic input/output system (“BIOS”) that controls certain basic functions of computing device 30. Random access memory (“RAM”) 36 and disk adapter 37 are also connected to system bus 32. It should be noted that software components including operating system 33 and application 34 may be loaded into RAM 36, which may be computing device's 30 main memory for execution. Disk adapter 37 may be an integrated drive electronics (“IDE”) adapter that communicates with a disk unit 38 (e.g., a disk drive). It is noted that the program for assessing a subject's vulnerability to developing at least one cardiometabolic-related condition, such as in connection with FIGS. 1A, 4-6, 8, 9A-9D, 10, 12-13, 14A-14B, 15, and 16A-16B, may reside in disk unit 38 or in application 34.

Computing device 30 may further include a communications adapter 39 connected to bus 32. Communications adapter 39 interconnects bus 32 with an outside network (e.g., wide area network) to communicate with other devices.

Aspects of the present disclosure are described herein with reference to flowchart illustrations and/or block diagrams of methods, apparatus (systems), and computing devices according to embodiments of the disclosure. It will be understood that computer-readable program instructions can implement each block of the flowchart illustrations and/or block diagrams, and combinations of blocks in the flowchart illustrations and/or block diagrams.

These computer-readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein includes an article of manufacture including instructions which implement aspects of the function/act specified in the flowchart and/or block diagram block or blocks. The computer-readable program instructions may also be loaded onto a computer, other programmable data processing apparatus, or other devices to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computing devices according to various embodiments of the present disclosure. In this regard, each block in the flowchart or block diagrams may represent a module, segment, or portion of instructions, which includes one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

### Applications and Advantages

The methods and systems of the present disclosure provide various advantages over existing diagnostic methods. For instance, in some embodiments, the systems and methods of the present disclosure identify metabolic problems earlier, thereby permitting an individual to achieve and maintain good cardiometabolic health before hidden tissue damage occurs. In particular, good metabolic health can help prevent the slow, years-to-decades irreversible damage to insulin-secreting beta cells that leads to prediabetes and diabetes. Likewise, good metabolic health can help prevent the slow, years-to-decades damage to the inner lining of arteries that can block blood flow to the heart, brain and other vital organs.

The methods and systems of the present disclosure can have various applications. For instance, in some embodiments, the methods and systems of the present disclosure can provide health care providers and the general public with a risk calculator that assesses cardiometabolic health and detects early metabolic imbalance (EMI), especially in subjects who do not meet the criteria for prediabetes or metabolic syndrome and elude conventional risk screening.

Additionally, the methods and systems of the present disclosure can be utilized through various platforms, such as a smart phone, a computer app, a web-based calculator, a clinical lab report, electronic medical records or normograms. Moreover, the methods and systems of the present disclosure can provide clinicians with a readily accessible, low-cost app that makes use of data routinely collected in clinical practice or health screening exams.

In addition, individuals monitoring their own health could utilize the methods and systems of the present disclosure to obtain the input data and use the app to assess their health status and disease risk. For instance, in some embodiments, the methods of the present disclosure may utilize shared decision making, where individuals are actively monitoring their cardiometabolic health status and engaged with their healthcare providers on how best to address less-than-optimal health and early risk factors.

### Additional Embodiments

Reference will now be made to more specific embodiments of the present disclosure and experimental results that provide support for such embodiments. However, Applicants note that the disclosure below is for illustrative purposes only and is not intended to limit the scope of the claimed subject matter in any way.

### Example 1. Early Metabolic Imbalance (EMI): Unveiling Hidden Diabetes Risk Among Apparently Healthy U.S. Young Adults

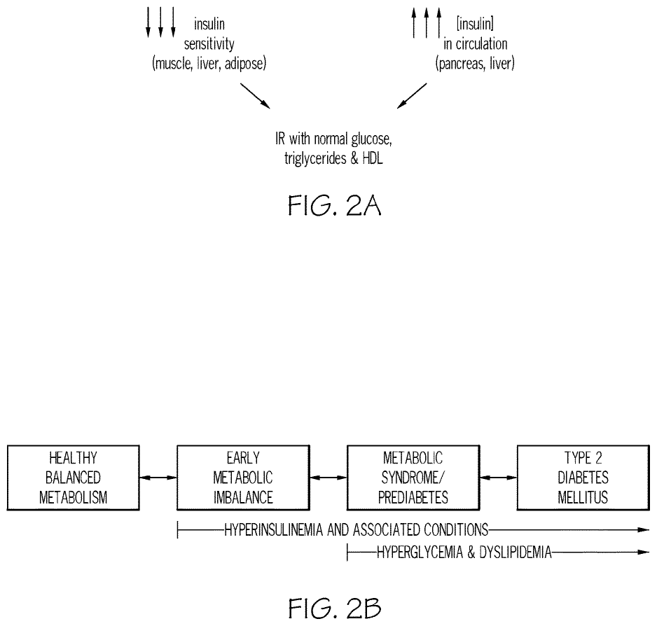

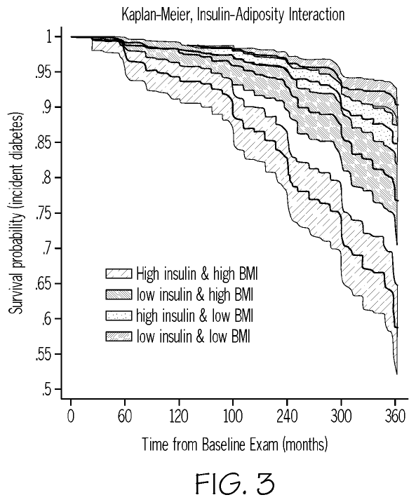

As illustrated in FIGS. 2A-2B, early metabolic imbalance (EMI) is a hidden state of compensated insulin resistance (IR). During EMI, beta-cell insulin secretion is generally intact and hyper-adaptive. Additionally, fasting glucose, fasting triglycerides (TG), high-density lipoprotein (HDL) and hemoglobin A1c levels are within normal limits during EMI. Moreover, EMI does not meet the criteria for prediabetes or metabolic syndrome. Furthermore, EMI eludes screening for diabetes and cardiovascular disease (CVD) risk.

EMI has multiple components that include insulin-adiposity interaction, hypoxemia, hypoxia, pro-inflammation, pro-coagulation, and pro-oxidation. However, screening for EMI presents an unmet need because EMI is prevalent in the U.S. population, especially among teens and young adults.

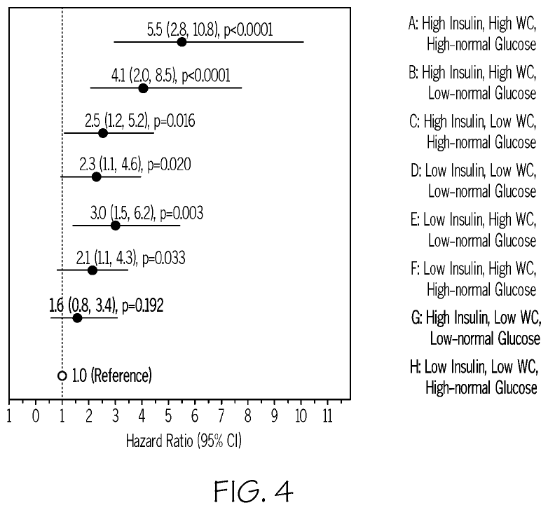

In this Example, Applicant aimed to determine whether, in addition to body mass index (BMI) and other canonical risk factors (e.g., FIG. 3), EMI components further increase the risk of future diabetes in apparently healthy young adults. In particular, Applicant aimed to determine whether hidden EMI components in apparently healthy young adults increase the risk for incident type 2 diabetes later in life. Additionally, for young adults without prediabetes, diabetes, metabolic syndrome or CVD, Applicant aimed to compare the prognostic power of Cox models with canonical diabetes risk factors to those that also include EMI components.

### Example 1.1. Design and Methods

Applicant performed a retrospective cohort analysis of the Coronary Artery Risk Development in Young Adults (CARDIA). Applicant applied the following exclusion criteria (all at baseline): (a) hyperglycemia, (b) hypertriglyceridemia, (c) low HDL-C, (d) pregnancy, (e) fasting <8 hours, and (f) diabetes or CVD.

The study included 3,292 participants with ages ranging from 18-30 at baseline (Table 1). Fasting insulin cut points were calibrated using time-dependent receiver operator characteristic curve (time ROC) analysis and stratified by BMI due to interacting variables.

TABLE 1
Study participants at baseline.

| Baseline characteristics | Mean (s.d.) or % | | Reference Range |
|---|---|---|---|
| age, years | 24.8 | (3.6) | n/a |
| sex, % female | 49.9 | | n/a |
| race, % Black | 52.2 | | n/a |
| fasting insulin, μIU/mL | 9.4 | (6.3) | see FIG. 3 legend |
| HOMA2-% S | 129.4 | (66.2) | ~100% |
| HOMA2-% B | 119.3 | (48.1) | ~100% |
| fasting glucose, mg/dL | 81 | (7.4) | <100 |
| fasting TG, mg/dL | 61.4 | (24.4) | <150 |
| HDL-C, mg/dL | 57.8 | (11.5) | >=50 (f), >=40 (m) |
| family history of diabetes, % | 13.5 | | n/a |
| fitness by treadmill, sec | 606.4 | (179.9) | n/a |
| serum uric acid, mg/dL | 5.2 | (1.3) | <=7.0 (f) <=8.0 (m) |
| white blood cells, ×109/L | 5.6 | (1.8) | 4.5-11 |
| high serum cotinine, % | 28.8 | | <15 ng/mL |
| BMI, kg/m² | 23.7 | (4.1) | <25 |

n = 3,292; n/a, not applicable; HOMA2, homeostatic model assessment of insulin resistance v.2; % S, percent insulin sensitivity; % B, percent beta-cell secretion; BMI, body-mass index.

The analysis was conducted using a stratified Cox proportional hazard regression using sex and race. The primary outcome was time to incident diabetes. The effect size was hazard ratio (HR) with 95% confidence limits (CI) and Harrell's c-statistic (prognostic power).

### Example 1.2. Results

The results are summarized in Tables 2-3 for Cox Model 1 (canonical risk factors, Table 2) and Cox Model 2 (Insulin-BMI category interaction and canonical risk factors, Table 3).

TABLE 2
Stratified Cox Regression, using sex/race,
with canonical risk factors.

| Baseline Predictor Variables | HR, Incident Diabetes | p-value |
|---|---|---|
| BMI >=25 vs. <25 | 2.0 (1.6, 2.4) | <0.0001 |
| family history of diabetes, | 1.9 (1.5, 2.4) | <0.0001 |
| 1stdegree rel. | | |
| poor fitness, treadmill test | 1.9 (1.5, 2.4) | <0.0001 |
| hypertension, >130/85 or medication | 1.7 (1.3, 2.3) | <0.0001 |
| high-normal glucose, >=82 vs. <82 | 1.4 (1.1, 1.7) | 0.002 |
| high serum cotinine, >=15 vs. <15 | 1.3 (1.0, 1.6) | 0.021 |

HR, hazard ratio; units and abbreviations as in Table 1.
Harrell's c-statistic for Model 1: 0.671

TABLE 3

| | HR (95% CI), | |
|---|---|---|
| Baseline Predictor Variables | Incident DM | p-value |
| high insulin & high BMI | 3.4 (2.5, 4.6) | <0.0001 |
| low insulin & high BMI | 1.6 (1.2, 2.2) | 0.004 |
| high insulin & low BMI | 1.4 (1.0, 1.8) | 0.041 |
| low insulin & low BMI | reference condition | — |
| family history of diabetes, | 1.9 (1.5, 2.5) | <0.0001 |
| 1^{st} degree relative | | |
| poor fitness, treadmill test | 1.7 (1.3, 2.3) | <0.0001 |
| hypertension, >130/85 or medication | 1.6 (1.2, 2.1) | 0.002 |
| high serum cotinine, >=15 vs. <15 | 1.4 (1.1, 1.8) | 0.002 |
| high-normal glucose, >=82 vs. <82 | 1.3 (1.0, 1.6) | 0.029 |

Stratified Cox Regression, using sex/race; abbreviations and units as in Tables 1 and 2.

### Example 2. Early Metabolic Imbalance (EMI) as a Hidden Risk Factor for Midlife Cardiovascular Disease

In this Example, Applicant aimed to determine if, beyond canonical risk factors, EMI components in apparently healthy young adults increase the risk of atherosclerotic cardiovascular disease (ASCVD). For young adults who do not meet the criteria for metabolic syndrome, prediabetes, diabetes, or CVD at baseline, Applicant compared the prognostic power of canonical CVD risk factors with and without EMI components.

The study design focused on a retrospective cohort analysis of the Coronary Artery Risk Development in Young Adults (CARDIA) study. Exclusion criteria included hyperglycemia, hypertriglyceridemia, low HDL-C, pregnancy, fasting <8 hours, diabetes or CVD, all at baseline.

The study included 3,292 participants, ages 18-30 at baseline. Table 4 summarizes the baseline characteristics of the study participants. Insulin, waist circumference and glucose cut points were calibrated using time-dependent receiver operator characteristic (time ROC) analysis. Insulin cut points were stratified due to interacting variables. The analysis included covariate-adjusted Cox proportional hazard regression models. The primary outcome included time to incident CVD as any fatal/non-fatal myocardial infarction, coronary revascularization, acute coronary syndrome, heart failure, stroke, transient ischemic attack, and carotid or peripheral artery disease. The effect size included hazard ratio (HR) with 95% confidence limits (CI) and Harrell's c-statistic (prognostic power).

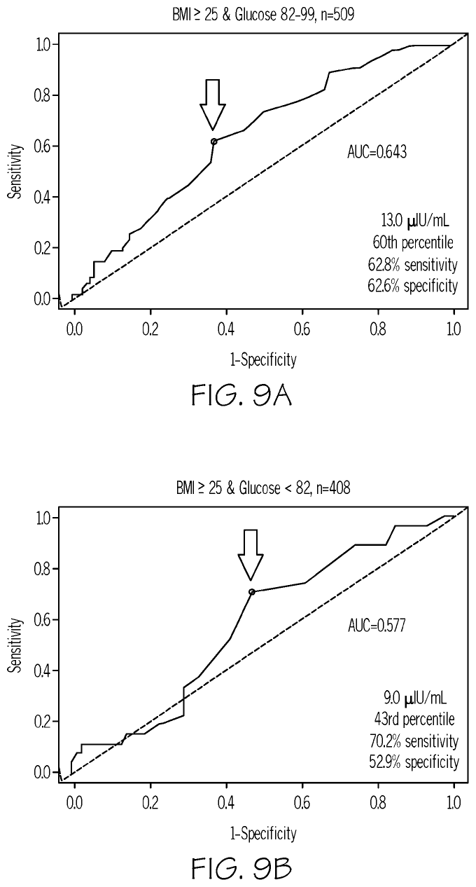

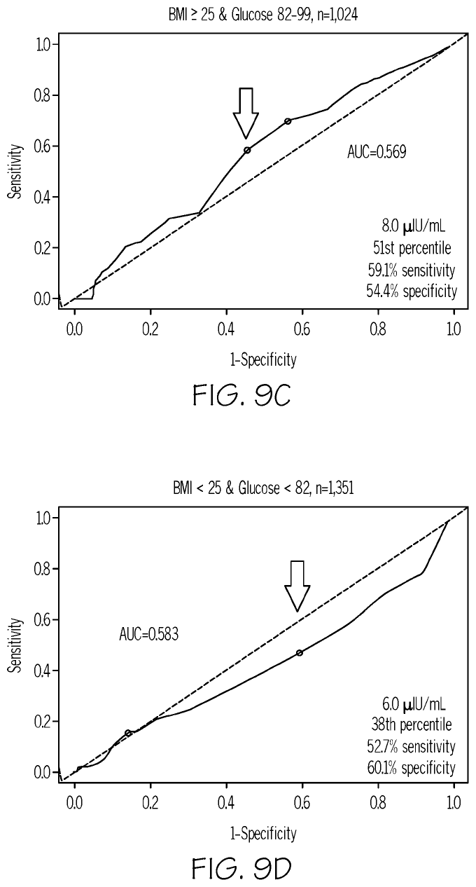

TABLE 4

| Baseline characteristics | Mean(s.d.) or % | | Reference Range | |
|---|---|---|---|---|
| age, years | 24.8 | (3.6) | n/a | |
| sex, % female | 49.9 | | n/a | |
| race, % Black | 52.2 | | n/a | |
| fasting insulin, mIU/L | 9.4 | (6.3) | see legends to FIGS. 8 and 9A-9D | |
| HOMA2-% S | 129.4 | (66.2) | ~100% | |
| HOMA2-% B | 119.3 | (48.1) | ~100% | |
| fasting glucose, mg/dL | 81 | (7.4) | <100 | |
| hypertension, % | 8.1 | | n/a | |
| LDL-C, mg/dL | 106.3 | (29.9) | <160 | |
| family Hx of CVD, % | 10.4 | | n/a | |
| fitness by treadmill, sec | 606.4 | (179.9) | n/a | |
| high serum cotinine, % | 28.8 | | <15 | ng/mL |
| GGT, U/L | 8.0 | (10.7) | <8 | U/L |

| | | | |
|---|---|---|---|
| platelet count, cells/μL | 263 K | (65) | 150 K-450 K |

| | | |
|---|---|---|
| waist circumference, cm | 80.2 (m), 71.9 (f) | 80.8 (m), 72.3 (f) |

Baseline characteristics of study participants, n = 3,292.
Abbreviations: n/a, not applicable; HOMA2, homeostatic model assessment of insulin resistance v.2; S, % insulin sensitivity; B, % β-cell secretion; GGT, gamma glutamyl transferase.

Table 5 summarizes the results for Cox Model 1 (ACC/AHA risk factors for CVD). Harrell's c-statistic for Model 1 was 0.713.

TABLE 5
Cox proportional hazards regression Model 1, which
includes the ACC/AHA canonical risk factors.

| Baseline Predictor Variables | HR (95% CI) | p-value |
|---|---|---|
| sex, male | 2.5 (1.7, 3.7) | <0.0001 |
| hypertension, >130/85 or medication | 2.0 (1.4, 3.0) | <0.0001 |
| increased waist circumference (WC) | 1.9 (1.4, 2.6) | <0.0001 |
| high serum cotinine, >=15 vs. <15 | 1.7 (1.2, 2.3) | 0.001 |
| high LDL-cholesterol, ≥160 mg/dL | 1.7 (1.0, 2.8) | 0.058 |
| family history, premature ASCVD | 1.5 (1.0, 2.2) | 0.071 |
| race, Black | 1.4 (1.0, 1.9) | 0.065 |
| poor fitness, treadmill test | 1.3 (1.1, 1.5) | 0.012 |
| low eGFR (2021 formula) | 0.8 (0.1, 5.5) | 0.792 |

HR, hazard ratio for incident CVD; CI, confidence interval); other units and abbreviations as in Table 1; for CARDIA subpopulation, n = 3,292.

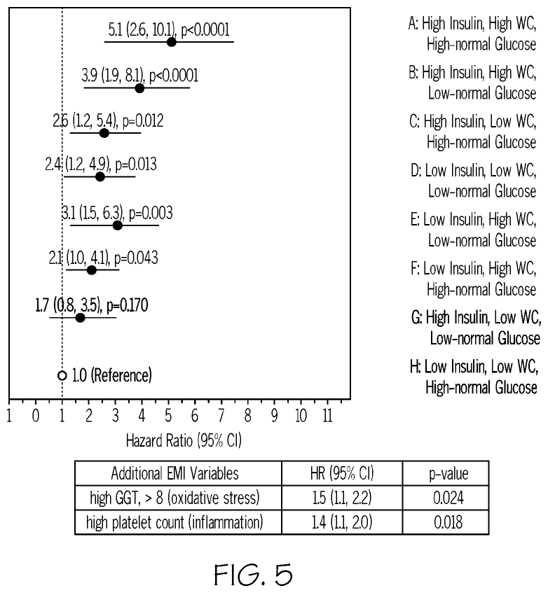

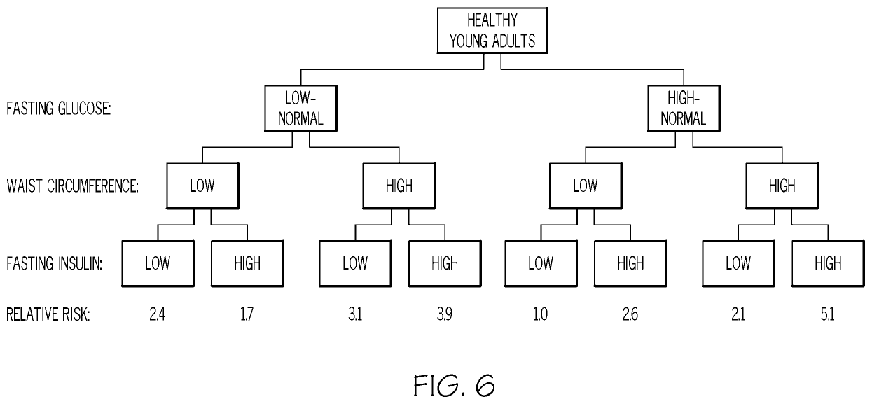

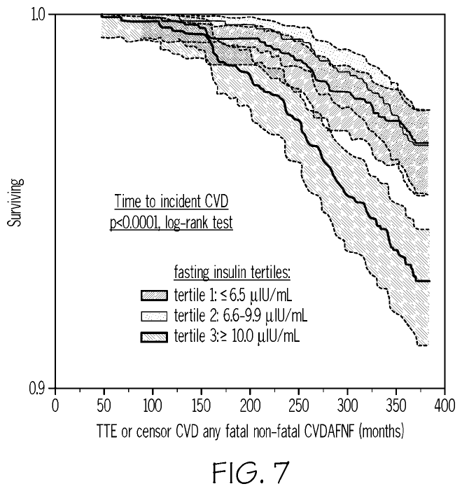

FIG. 4 summarizes the results for Cox Model 2 (ACC/AHA risk factors for plus Insulin-WC Glucose Categorical Interaction). Harrell's c-statistic for Model 2 was 0.728. FIG. 5 summarizes the results for Cox Model 3 (Model 2 plus additional EMI variables). Harrell's c-statistic for Model 3 was 0.740. FIG. 6 summarizes the criteria for assessing CVD risk in healthy young adults.

In summary, hidden EMI in apparently healthy young adults in CARDIA was a multi-component risk factor for future CVD. The interaction among EMI components, particularly fasting insulin, waist circumference and normal-range glucose, was critical for explaining the hidden risk of CVD later in life. Cox models that included EMI components had more prognostic power for assessing CVD risk compared to those with canonical risk factors alone.

### Example 3. Prognostic Cut Points of Fasting Insulin in Apparently Healthy Young Adults for the Development of Future Cardiovascular Disease

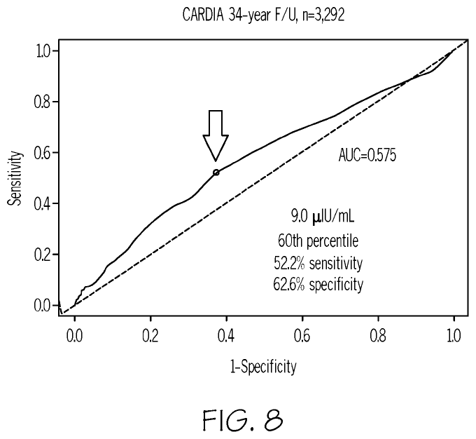

High fasting insulin in apparently healthy young adults is a risk factor for future CVD. In fact, normoglycemic young adults with fasting insulin above the top tertile appear to be at increased risk for midlife CVD (FIG. 7). Rather than tertiles, this Example aimed to better calibrate prognostic insulin cut points using receiver operator characteristic curve (ROC) analysis.

In particular, this Example used a time-ROC analysis of data from a healthy young adult population to calibrate a cut point optimum of fasting insulin for predicting risk for future CVD. This Example also aimed to conduct a stratified time-ROC analysis to account for the interactions and effect modification of fasting insulin by waist circumference and fasting glucose. Additionally, this Example applied the cut point optima to a Cox analysis of incident CVD.

### Example 3.1. Study Design and Methods

Applicant performed a retrospective cohort analysis of CARDIA and applied the following exclusion criteria (all at baseline): pregnancy, hyperglycemia, hypertriglyceridemia, low HDL, diabetes, CVD or fasting <8 hours. The number of subjects were 3,292, with ages ranging from 18-30 years at baseline, with a 35-year mean follow up (Table 6).

TABLE 6

| Baseline characteristics | Mean(s.d.) or % | | Reference Range |
|---|---|---|---|
| age, years | 24.8 | (3.6) | n/a |
| sex, % female | 49.9 | | n/a |
| race, % Black | 52.2 | | n/a |
| fasting insulin, mIU/mL | 9.4 | (6.3) | see legends to FIGS. 8 |
| | | | and 9A-9D |
| fasting glucose, mg/dL | 81.0 | (7.4) | <100 |
| HOMA2-% S | 129.4 | (66.2) | ~100% |
| HOMA2-% B | 119.3 | (48.1) | ~100% |
| high LDLc, % | 4.9 | | <160 |
| fasting TG, mg/dL | 61.4 | (24.4) | <150 |
| HDL-C, mg/dL | 57.8 | (11.5) | >=50 (f), >=40 (m) |
| family Hx of ASCVD, % | 10.4 | | n/a |
| fitness by treadmill, sec | 606.4 | (179.9) | n/a |
| hypertension, % | 5.2 | (1.3) | >135/>80 mmHg |
| low eGFR, % | 0.9 | | <60 mL/min/1.73 m² |
| high serum cotinine, % | 28.8 | | <15 ng/mL |
| BMI, kg/m² | 23.7 | (4.1) | <25 |

Baseline characteristics of CARDIA participants, n = 3,292.
abbreviations: n/a, not applicable; HOMA2, homeostatic model assessment of insulin resistance v.2; % S, percent insulin sensitivity; % B, percent beta-cell insulin secretion; BMI, body-mass index.

Time-ROC analysis was performed by unadjusted and covariate-adjusted Cox with inverse probability weighting (‘time ROC’ package in R v4.2.1). The insulin cut point optimum was defined by shortest distance to ideal discrimination point: [0,1] (x, y coordinate). The covariates included canonical ACC/AHA risk factors (FIG. 8 and legend).

Time-ROC analyses were stratified by BMI (<25 vs ≥25 kg/m²) and fasting glucose (<82 vs ≥82 mg/dL) due to effect modification (i.e., interaction between insulin, adiposity and glucose). Substituting waist circumference for BMI provided similar results. Forest plot of Cox proportional hazard ratios were used to compare states of 3-way interaction between insulin, glucose, and BMI.

### Example 3.2. Results and Discussion

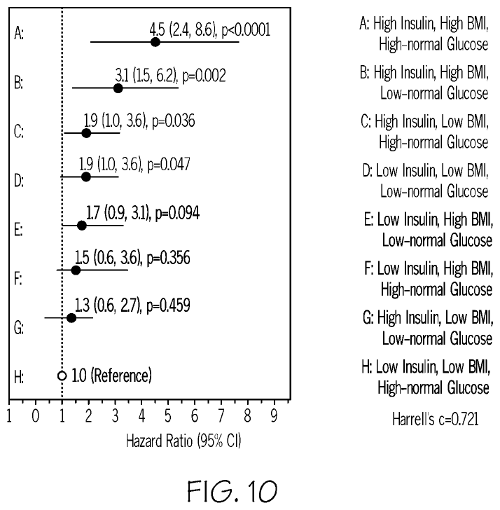

The results are shown in FIGS. 8, 9A-9D, and 10. FIG. 8 shows the time-ROC curve for fasting insulin. FIGS. 9A-9D show time-ROC curves for fasting insulin, which were stratified by BMI & fasting glucose. FIG. 10 shows Cox hazard ratios utilizing time-ROC cut points.

In sum, this Example illustrates that fasting serum insulin has prognostic power to estimate the risk of future CVD in apparently healthy young adults without prediabetes or metabolic syndrome. Insulin cut point optima and the risk of CVD by insulin are modified by BMI and glucose.

### Example 4. Prognostic Fasting Insulin Cut Points for Hidden Diabetes Risk Among Apparently Healthy Young Adults: CARDIA 30-Year Follow Up

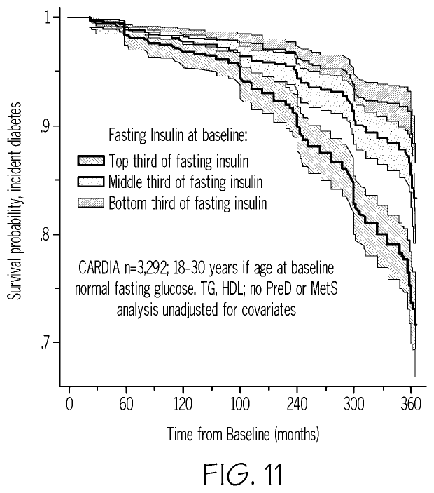

This Example considers insulin cut points for diabetes risk. Apparently healthy young adults with fasting insulin above the top tertile are at increased risk for diabetes later in life (FIG. 11). Rather than tertiles, it is better to calibrate prognostic cut points from time-dependent receiver operator characteristic curve (time ROC) analysis of survival data. In this Example, Applicant aimed to determine if cut point optima for fasting insulin can be calibrated using a time-ROC analysis of the CARDIA study.

### Example 4.1. Methods

The inclusion criteria for a retrospective cohort analysis of CARDIA include prior study participants. The exclusion criteria included pregnancy, hyperglycemia, hypertriglyceridemia, low HDL, diabetes, and CVD or fasting <8 hours, all at baseline. The number of participants included 3,292, ages 18-30 years at baseline with a 30-year mean follow up (Table 7).

TABLE 7

| Baseline characteristics | Mean(s.d.) or % | | Reference Range |
|---|---|---|---|
| age, years | 24.8 | (3.6) | n/a |
| sex, % female | 49.9 | | n/a |
| race, % Black | 52.2 | | n/a |
| fasting insulin, mIU/L | 9.4 | (6.3) | See legends to FIGS. |
| | | | 12-16A-16B |
| HOMA2-% S | 129.4 | (66.2) | ~100% |
| HOMA2-% B | 119.3 | (48.1) | ~100% |
| fasting glucose, mg/dL | 81 | (7.4) | <100 |
| fasting TG, mg/dL | 61.4 | (24.4) | <150 |
| HDL-C, mg/dL | 57.8 | (11.5) | >=50 (f), >=40 (m) |
| family Hx of diabetes, % | 13.5 | | n/a |
| fitness by treadmill, sec | 606.4 | (179.9) | n/a |
| serum uric acid, mg/dL | 5.2 | (1.3) | <=7.0 (f) <=8.0 (m) |
| white blood cells, ×109/L | 5.6 | (1.8) | 4.5-11 |
| high serum cotinine, % | 28.8 | | <15 ng/mL |
| BMI, kg/m2 | 23.7 | (4.1) | <25 |

Study participants at baseline, n = 3,292.
n/a, not applicable; HOMA2, homeostatic model assessment of insulin resistance v.2; % S, percent insulin sensitivity; % B, percent beta-cell secretion; BMI, body-mass index.

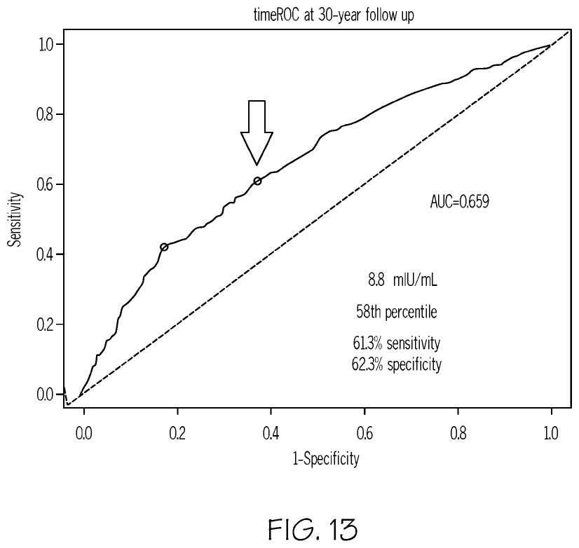

For the timeROC analysis, unadjusted and covariate-adjusted Cox with inverse probability weighting were used (‘time ROC’ package in R v4.2.1). For the covariates, canonical ADA risk factors (see FIG. 13 legend) plus serum cotinine, uric acid, TG/HDL ratio and WBC count were used.

The insulin cut point optimum was defined by the shortest distance to ideal discrimination point ([0,1], x, y coordinate). The analyses were stratified by BMI (<25 vs ≥25 kg/m²) because of the effect of modification (i.e., interaction between insulin and adiposity). Substituting waist circumference for BMI gave similar results. An original insulin antibody cross-reacted with pro-insulin. However, a newer insulin assay was specific for insulin. The correlation between the two insulin assays was high (R=0.8).

### Example 4.2. Results

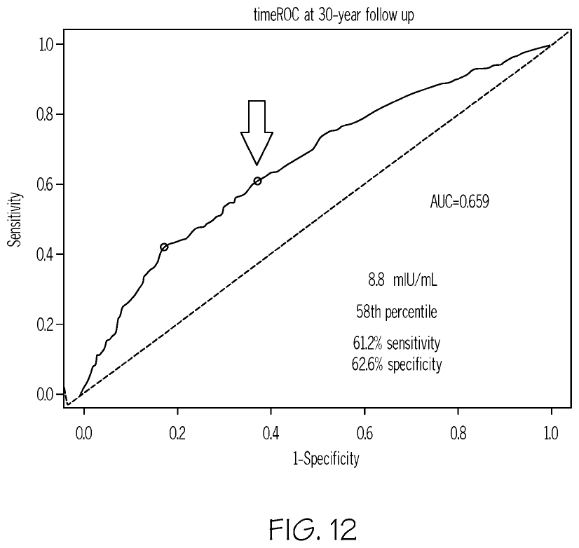

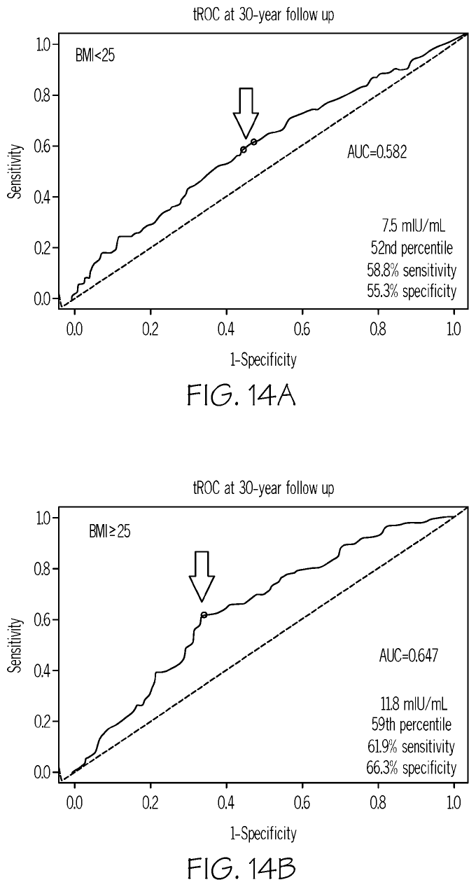

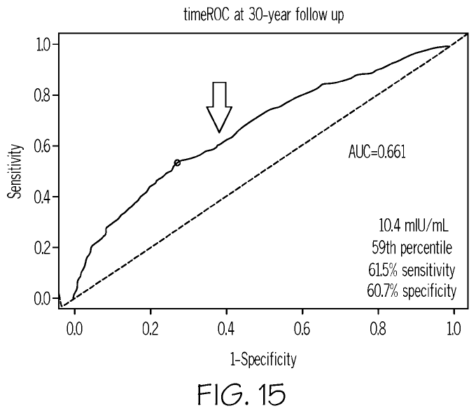

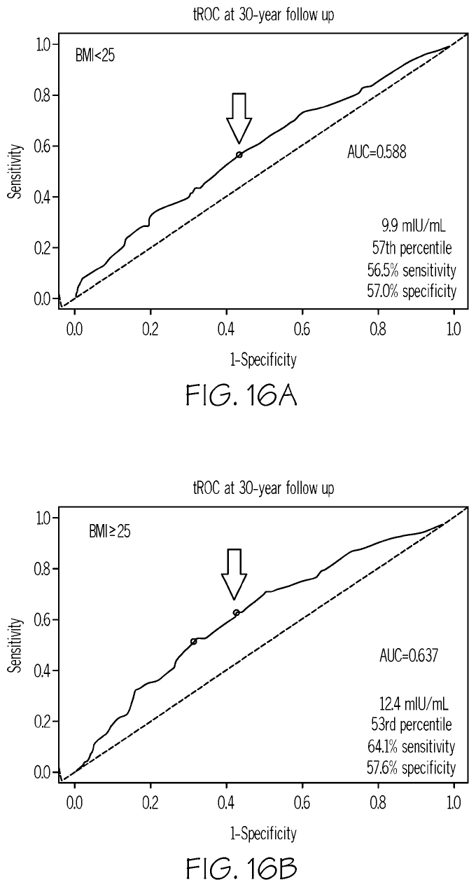

The results are summarized in FIGS. 12-13, 14A-14B, 15, and 16A-16B. FIG. 12 shows unadjusted time-ROC analysis for fasting insulin. FIG. 13 shows a covariate-adjusted time-ROC analysis. FIGS. 14A-14B show time-ROC analysis stratified by BMI. The stratification shows that the insulin cut point optimum and prognostic power are higher in those with BMI ≥25. FIG. 15 shows a time-ROC analysis with a covariate-adjusted, newer insulin assay. FIGS. 16A-16B show a time-ROC analysis with a newer insulin assay stratified by BMI.

The results indicate that fasting serum insulin has prognostic power for assessing diabetes risk in healthy young adults without prediabetes or metabolic syndrome. The insulin cut point optimum is modified by BMI and falls between the median and top tertile.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A method of assessing a subject's vulnerability to developing at least one cardiometabolic-related condition, said method comprising:
receiving a plurality of health-related data of the subject,
calculating a risk score from the plurality of health-related data; and
correlating the risk score to the subject's vulnerability to the at least one cardiometabolic-related condition.

2. The method of claim 1, wherein the plurality of health-related data are selected from the group consisting of demographic data, age, gender assigned at birth, race, socio-economic history, medical history, family history of diabetes, family history of cardiovascular disease, clinical data, vital signs, laboratory test results, blood test results, imaging test results, non-invasive health measurements, body-mass index (BMI), waist circumference, waist circumference-to-height ratio, fitness level, Fitbit activity, electrocardiogram (EKG) profile, pulse oximetry data, blood gas parameters, blood pressure, hypertension, body composition measurements, inflammatory marker levels, C-reactive protein levels, platelet levels, white blood cell (WBC) levels, oxidative stress marker levels, tissue hypoxia marker levels, gamma glutamyl transferase (GGT) levels, aspartate aminotransferase (AST) levels, alanine aminotransferase (ALT) levels, bilirubin levels, lactic acid levels, lactate levels, uric acid levels, urate levels, uric acid/creatinine ratio, cotinine levels, insulin resistance measures, beta cell insulin secretion levels, insulin levels, fasting insulin levels, non-fasting insulin levels, fasting c-peptide levels, non-fasting c-peptide levels, glucose levels, fasting glucose levels, non-fasting glucose levels, hemoglobin A1c levels, triglyceride (TG) levels, fasting TG levels, non-fasting TG levels, high-density lipoprotein cholesterol (HDL-C) levels, TG/HDL ratio, low-density lipoprotein cholesterol (LDL-C) levels, non-HDL-C levels, oxidized LDL-C levels, apolipoprotein B levels, apolipoprotein C-III levels, physical fitness measures, and combinations thereof.

3. (canceled)

4. The method of claim 1, wherein the plurality of health-related data comprise fasting insulin or fasting c-peptide levels, fasting glucose or hemoglobin A1c levels, cotinine levels, body-mass index (BMI), fitness level, hypertension, and family history of diabetes.

5. (canceled)

6. The method of claim 1, wherein the plurality of health-related data comprise fasting insulin or fasting c-peptide levels, fasting glucose or hemoglobin A1c levels, waist circumference or body-mass index (BMI) or waist circumference-to-height ratio, platelet levels, and gamma glutamyl transferase (GGT) levels.

7. (canceled)

8. The method of claim 1, wherein the plurality of health-related data comprise family history of diabetes, race, gender assigned at birth, age, fasting glucose or hemoglobin A1c levels, fasting insulin or fasting c-peptide levels, physical fitness measures, hypertension, cotinine levels, uric acid levels, TG/HDL ratio, and white blood cell (WBC) levels.

9. The method of claim 1, wherein the risk score is calculated based on the subject's fasting insulin or fasting c-peptide levels and fasting glucose or hemoglobin A1c levels.

10. The method of claim 1, wherein the at least one cardiometabolic-related condition is selected from the group consisting of early metabolic imbalance (EMI), prediabetes, hyperinsulinemia, compensatory hyperinsulinemia, metabolic syndrome, insulin resistance syndrome, early metabolic dysregulation, diabetes, type 2 diabetes, gestational diabetes, latent autoimmune diabetes of adults, monogenic forms of diabetes, type 1 diabetes, cardiovascular disease (CVD), atherosclerotic cardiovascular disease (ASCVD), heart attack, stroke, peripheral vascular disease, insulin resistance, oxidative stress, subclinical inflammation, hypoxemia, subclinical hypoxemia, hypoxia, subclinical hypoxia, pre-conditions thereof, and combinations thereof.

11-15. (canceled)

16. The method of claim 1, wherein the subject is a human being.

17. The method of claim 1, wherein the subject is not suffering from or diagnosed with at least one cardiometabolic-related condition.

18. (canceled)

19. The method of claim 1, further comprising a step of making a treatment decision based on the subject's vulnerability to the at least one cardiometabolic-related condition.

20. The method of claim 19, wherein the treatment decision comprises monitoring the subject for signs or symptoms of the at least one cardiometabolic-related condition, monitoring the subject for pre-conditions or risk factors of the at least one cardiometabolic-related condition, administering a therapeutic agent to the subject, and combinations thereof.

21-22. (canceled)

23. The method of claim 19, wherein the treatment decision comprises administering a therapeutic agent or intervention to the subject, wherein the therapeutic intervention is selected from the group consisting of a nutritional program, a physical activity program, a weight-loss program, administration of one or more nutritional supplements, a non-pharmaceutical intervention, administration of one or more pharmaceutical agents, and combinations thereof.

24. (canceled)

25. The method of claim 1, wherein the method occurs through utilization of a manual health and risk score calculator.

26. The method of claim 25, wherein the manual health and risk score calculator is in the form of a fillable questionnaire.

27. The method of claim 1, wherein the method occurs through the utilization of a computing device, wherein the receiving comprises entering the health-related data to the computing device, wherein the calculating of the risk score occurs by the computing device, and wherein the correlating comprises generating an output from the computing device.

28. The method of claim 27, wherein the computing device comprises a web-based program, an application-based program, and combinations thereof.

29. (canceled)

30. The method of claim 27, wherein the computing device comprises a machine-learning algorithm trained on the plurality of health-related data.

31. A system for assessing a subject's vulnerability to developing at least one cardiometabolic-related condition, said system comprising:
instructions for receiving a plurality of health-related data of the subject,
instructions for calculating a risk score from the plurality of health-related data; and
instructions for correlating the risk score to the subject's vulnerability to at least one cardiometabolic-related condition.

32. The system of claim 31, further comprising instructions for making a treatment decision based on the subject's vulnerability to the at least one cardiometabolic-related condition, wherein the treatment decision instructions comprise monitoring the subject for signs or symptoms of the at least one cardiometabolic-related condition, monitoring the subject for pre-conditions or risk factors of the at least one cardiometabolic-related condition, administering a therapeutic intervention or agent to the subject, and combinations thereof.

33. (canceled)

34. The system of claim 31, wherein the instructions comprise a manual health and risk score calculator.

35. The system of claim 34, wherein the manual health and risk score calculator is in the form of a fillable questionnaire.

36. The system of claim 31, wherein the system comprises a computing device, and wherein the computing device comprises programming instructions for receiving the plurality of health-related data of the subject, programming instructions for calculating the risk score from the plurality of health-related data; and programming instructions for correlating the risk score to the subject's vulnerability to the at least one cardiometabolic-related condition.

37. The system of claim 36, wherein the computing device comprises a web-based program, an application-based program, and combinations thereof.

38. The system of claim 36, wherein the computing device comprises a machine-learning algorithm trained on the plurality of health-related data.

39-44. (canceled)
